(19) United States
(12) Patent Application Publication
(10) Pub. No.: US 2026/0076656 A1
(43) Pub. Date: Mar. 19, 2026

(54) Universal Tool Adapter for Image-Guided Surgery

(71) Applicant: AUGMEDICS LTD., Yokneam (IL)

(72) Inventors: Stuart Wolf, Yokneam (IL); Nissan Elimelech, Beerotaim (IL); Tomer Gera, Kfar Tavor (IL)

(21) Appl. No.: 19/397,629

(22) Filed: Nov. 21, 2025

Related U.S. Application Data
(63) Continuation of application No. 18/044,380, now Pat. No. 12,502,163.
Application No. 18/044,380 is a continuation-in-part of application No. 17/015,199, filed on Sep. 9, 2020, now Pat. No. 12,239,385.

(51) Int. Cl. A61B 17/00; A61B 90/00

(52) U.S. Cl. CPC A61B 17/00; A61B 90/03; A61B 90/39; A61B 2017/00486; A61B 2090/031; A61B 2090/3937; A61B 2090/3983

(57) ABSTRACT

A tool adapter (18), consisting of an adapter arm (102) having a proximal end terminating with a connection (110) and a distal end having a circular coupling (104), the circular coupling having a center and defining an axis (114) orthogonal to the circular coupling and passing through the center. The adapter also has a tool grip (32) rotatingly connected to the circular coupling so as to permit rotation of the tool grip about the axis, the tool grip being configured to fixedly retain a tool (16) along the axis.

### CROSS-REFERENCE TO RELATED APPLICATIONS

This Application is a continuation under 35 U.S. C. § 120 of U.S. patent application Ser. No. 18/044,380, filed on Mar. 8, 2023, which is a national stage entry under 35 C.F. R. § 371 of PCT Application No. PCT/IB2021/058088, filed on Sep. 5, 2021, which is a continuation in-part of U.S. patent application Ser. No. 17/015,199, filed on Sep. 9, 2020 and issued as U.S. Pat. No. 12,239,385 on Mar. 4, 2025, the entire contents of each of which are hereby incorporated by reference.

### FIELD

The present disclosure relates generally to surgery, and specifically to a tool adapter used to hold a tool during image-guided surgery.

### BACKGROUND

During surgery, it is often necessary to have a tool used in the surgery held, typically in a relatively fixed position. While the surgeon performing the surgery may hold the tool, having the tool held mechanically frees up the surgeon's hands for other tasks. During image-guided surgery, it is often necessary to track or determine a position of a tool used in the surgery with respect to the patient anatomy with a desired accuracy.

An image of the tool may be then displayed to the surgeon with respect to an image of the anatomy of the patient (e.g., augmented on the patient image) based on the tool tracked location.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

### SUMMARY

An embodiment of the present disclosure provides a tool adapter, consisting of: an adapter arm having a proximal end terminating with a connection and a distal end having a circular coupling, the circular coupling having a center and defining an axis orthogonal to the circular coupling and passing through the center; and a tool grip rotatingly connected to the circular coupling so as to permit rotation of the tool grip about the axis, the tool grip being configured to fixedly retain a tool along the axis.

In a disclosed embodiment the tool grip includes a collet housed in a collet holder, and the collet is configured on compression to grip the tool. The collet holder may be held by the circular opening, and may be configured to be unable to translate along the axis. The tool adapter may include a collet fastening head configured to screw onto the collet holder so as implement the compression of the collet.

In a further disclosed embodiment the tool adapter includes a position marker fixedly connected to the connection of the proximal end in a preset spatial relationship with the axis, the marker having optical elements enabling the marker to be tracked spatially, so that tracking of the marker provides tracking of the tool retained by the tool grip.

In a yet further disclosed embodiment the tool adapter includes a torque limiting device coupled to the tool grip and configured to apply a preset threshold torque to the grip so that rotation of the tool grip is permitted when the threshold torque is exceeded. The torque limiting device may consist of a plurality of pins retained within the tool grip and configured to exert force against the circular opening.

In an additional disclosed embodiment the tool adapter includes a total indicated runout (TIR) controller coupled to the tool grip and configured to apply a pre-determined TIR to the tool.

There is further provided, according to an embodiment of the present disclosure, a method for conducting image guided surgery, consisting of: providing an adapter arm having a proximal end terminating with a connection and a distal end having a circular coupling, the circular coupling having a center and defining an axis orthogonal to the circular coupling and passing through the center; rotatingly connecting a tool grip to the circular coupling so as to permit rotation of the tool grip about the axis, the tool grip being configured to fixedly retain a tool along the axis; fixedly connecting a position marker to the connection of the proximal end of the adapter arm in a preset spatial relationship with the axis, the position marker including optical elements enabling the marker to be tracked spatially, so that tracking of the position marker provides tracking of the tool fixed to the tool grip; and presenting an image of the tracked tool to a professional performing the surgery.

There is further provided, according to an embodiment of the present disclosure, a method consisting of: fixedly connecting a position marker to a connection of a proximal end of an adapter arm, the adapter arm further having a distal end including a circular coupling, the circular coupling having a center and defining an axis orthogonal to the circular coupling and passing through the center, wherein the connection is in a preset spatial relationship with the axis; inserting a tool through a tool grip of the adapter arm, wherein the tool grip includes a collet and is rotatingly connected to the circular coupling so as to permit rotation of the tool grip about the axis, the tool grip being configured to fixedly retain the tool along the axis; and compressing the collet, wherein the collet is configured on compression to grip the tool.

In a disclosed embodiment the method includes positioning a termination of the tool on a predefined location to allow calibration of the tool. The method may also include inserting the tool into a patient while the marker and tool are tracked.

In a further disclosed embodiment the compressing of the collet includes rotating a collet fastening head.

In a yet further disclosed embodiment the position marker includes optical elements enabling the marker to be tracked spatially, so that tracking of the position marker provides tracking of the tool fixed to the adapter arm.

The present disclosure will be more fully understood from the following detailed description of the embodiments thereof, taken together with the drawings, in which:

### BRIEF DESCRIPTION OF THE DRAWINGS

FIG. 1 schematically illustrates use of a tool adapter in an augmented reality system, according to an embodiment of the present disclosure;

FIG. 2A is a schematic diagram illustrating an augmented reality assembly, according to an embodiment of the present disclosure;

FIG. 2B is a schematic diagram illustrating a head-up display, according to another embodiment of the present disclosure;

FIG. 3 is a schematic diagram illustrating an exemplary tool adapter and a tool, according to an embodiment of the present disclosure;

FIG. 4 is a schematic exploded view of the tool adapter and the tool, according to an embodiment of the present disclosure;

FIG. 5 is a schematic cross-section of the tool adapter and the tool, according to an embodiment of the present disclosure;

FIG. 6 is a schematic diagram illustrating an exemplary tool adapter and a tool, according to another embodiment of the present disclosure; and

FIG. 7 is a flowchart of steps performed in using a tool adapter with the augmented reality system, according to an embodiment of the present disclosure.

### DETAILED DESCRIPTION OF EMBODIMENTS

### Overview

During a medical procedure performed using an augmented reality system, a tool used in the procedure, such as a screwdriver used for pedicle screws, typically needs to be tracked, so that images presented to a medical professional using the system correctly register with the tool and with the patient undergoing the procedure. In order to track the tool, a marker is typically connected to the tool, and the marker is tracked by the system. However, in the case of tools such as screwdrivers that need one or more rotations, or even a partial rotation, the marker must stay within the field of view of the tracking system so that tracking is maintained. Furthermore, to eliminate tracking errors, total indicated runout should be minimized. Furthermore, whether rotating tools or fixed tools (i.e., tools which are typically not rotated during use, such as a probe, a pointer or a pilot) are used, the application of a firm gripping force via the adapter, holding the tool in place with respect to the marker, is desired.

Embodiments of the present disclosure provide a solution that overcomes both of these problems by attaching a tool adapter, having the marker, to the tool, and configuring the tool adapter to be able to rotate about the tool without changing the spatial relationship of the tool to the marker.

The tool adapter comprises an adapter arm which terminates at a proximal end in a connection to the marker, and at a distal end in a circular opening. A tool grip is rotatingly connected to the circular opening so as to permit rotation of the tool grip about an axis defined by the opening, the tool grip being configured to fixedly retain a tool along the axis.

The tool grip typically comprises a collet housed in a collet holder, and the collet is configured to grip the tool when the collet is compressed. Use of a collet firmly and stably holds the tool in a centered location. The collet holder is held in the circular opening, and the combination of the collet, collet holder, and circular opening means that the shaft of the tool remains accurately along the axis of the opening during rotation, and that there is very low total indicated runout (TIR) during all rotations. The inventors have found that in contrast to prior art systems that have a TIR of the order of 100 microns, embodiments of the present disclosure have a TIR of the order of 10 microns, and thus substantially improve the accuracy of tracking of a gripped tool.

### System Description

In the following, all directional references (e.g., upper, lower, upward, downward, left, right, top, bottom, above, below, vertical, and horizontal) are only used for identification purposes to aid the reader's understanding of the present disclosure, and do not create limitations, particularly as to the position, orientation, or use of embodiments of the disclosure.

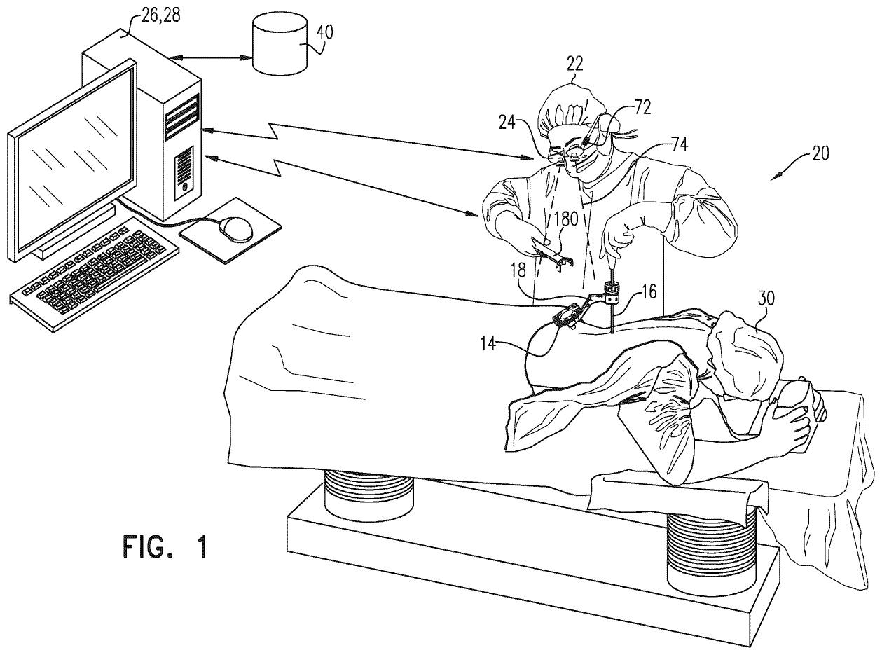

Reference is now made to FIG. 1, which schematically illustrates use of a tool adapter 18 in an augmented reality system 20, according to an embodiment of the present disclosure. By way of example and for simplicity, in the following description system 20 is assumed to be used by a medical professional 22 in a medical procedure wherein adapter 18 is attached to a cylindrical tool 16, for example a screwdriver, using a wrench 180, as is explained in more detail below. While the description herein assumes a medical procedure, it will be understood that embodiments of the present disclosure may be used in non-medical situations.

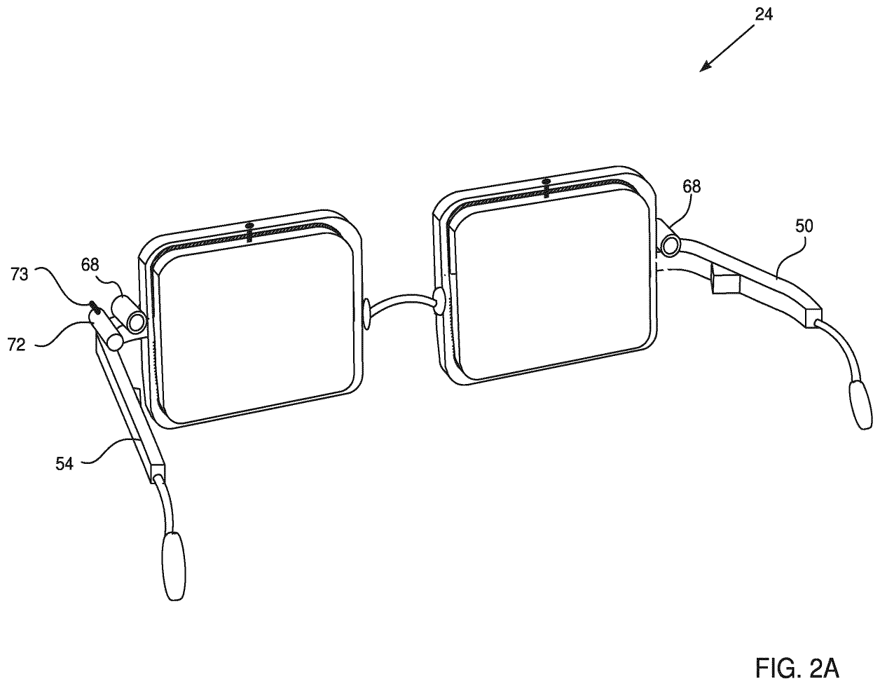

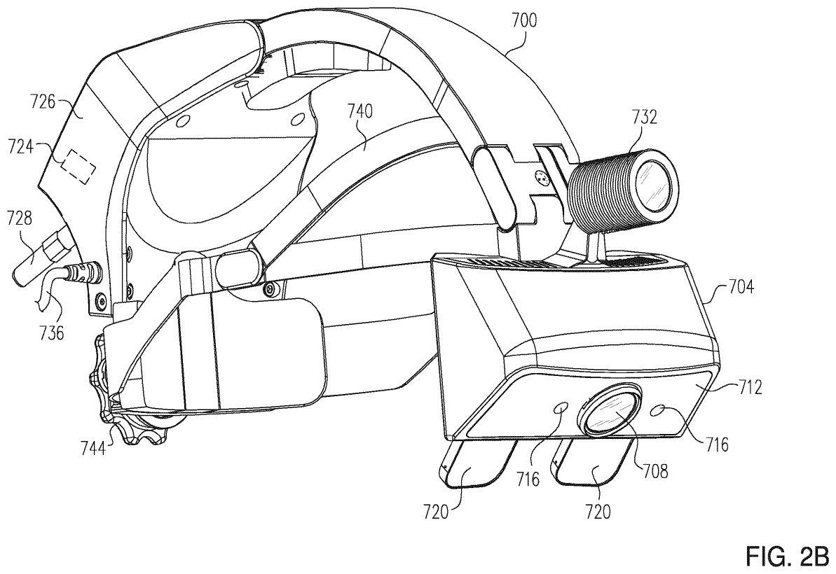

System 20 is operated by medical professional 22, who wears, by way of example, an augmented reality assembly 24, described in more detail below with respect to FIG. 2A, which tracks tool adapter 18. It will be understood that assembly 24 is but one type of tracking system that is able to track the tool holder, and that the scope of the present disclosure comprises any tracking system able to track tool adapter 18 and/or a position marker 14 mounted thereon.

Assembly 24 comprises, inter alia, an image capturing device 72, also termed herein a camera 72, that has a field of view indicated by a line of sight 74 and that is configured to capture images in the visible spectrum and/or in the non-visible spectrum, such as the Infra-Red (IR) spectrum. Assembly 24 and functions of system 20, processor 26, and device 72 are described below. An assembly similar to augmented reality assembly 24, and its operation, are described in U.S. Pat. No. 9,928,629, to Benishti, et al., whose disclosure is incorporated herein by reference.

While assembly 24 may be incorporated for wearing into a number of different retaining structures on professional 22, in the present Figure the retaining structure is assumed to be similar to a pair of spectacles. Those having ordinary skill in the augmented reality art will be aware of other possible structures, such as incorporation of the augmented reality assembly into a head-up display that is integrated into a headset worn by the user of system 20, and all such structures are assumed to be comprised within the scope of the present disclosure. Another exemplary head-up display is described below, with reference to FIG. 2B.

System 20 comprises and is under overall control of a processor 26. In one embodiment processor 26 is assumed to be incorporated within a stand-alone computer 28, and the processor typically communicates with other elements of the system, including assembly 24, wirelessly, as is illustrated in FIG. 1. Alternatively or additionally, processor 26 may use optical and/or conducting cables for communication. In further alternative embodiments processor 26 is integrated within assembly 24, or in the mounting of the assembly. Processor 26 is typically able to access a database 40, wherein are stored images and other visual elements used by system 20. Software enabling processor 26 to operate system 20 may be downloaded to the processor in electronic form, over a network, for example. Alternatively or additionally, the software may be provided on non-transitory tangible media, such as optical, magnetic, or electronic storage media.

The medical procedure exemplified here is on a patient 30, and during the procedure the position marker 14 is incorporated into tool adapter 18. As described below, marker 14 is trackable by processor 26, so that the processor is able to track also the tool adapter to which the marker is attached, and a tool 16 held by the adapter.

FIG. 2A is a schematic diagram illustrating assembly 24, according to an embodiment of the present disclosure. As stated above, assembly 24 is configured, by way of example, as a pair of spectacles 50 mounted on a frame 54.

At least one image capturing device 68 and/or 72 is attached to frame 54. Typically, devices 68 and/or 72 comprise cameras configured to capture images of scenes viewed by the professional's eyes, including images of marker 14 in the visible spectrum and/or non-visible spectrum.

As stated above assembly 24 comprises camera 72, which is configured to capture images of elements of a scene, including marker 14, in front of assembly 24. The images are produced from radiation projected by a projector 73 that is in the spectrum detected by camera 72. Projector 73 is located in close proximity to camera 72, so that radiation from the projector, that has been retroreflected, is captured by camera 72. The camera typically has a bandpass filter configured to block other radiation, such as that projected by surgical lighting. Typically, camera 72 and projector 73 operate in a non-visible region of the spectrum, such as in the near infra-red spectrum. As is described below, at least some retroreflected radiation is typically received from marker 14, and processor 26 uses the image of the marker produced by camera 72 from the received radiation to track the marker, and thus the position and orientation of adapter 18 and of tool 16.

FIG. 2B is a schematic figure illustrating a head-up display (HUD) 700, according to an embodiment of the present disclosure. HUD 700 is worn by professional 22, and may be used in place of assembly 24 (FIG. 1). HUD 700 comprises an optics housing 704 which incorporates an infrared camera 708. Housing 704 also comprises an infrared transparent window 712, and within the housing, i.e., behind the window, are mounted one or more infrared projectors 716. Mounted on housing 704 are a pair of augmented reality displays 720, which allow professional 22 to view entities, such as part or all of patient 30 through the displays, and which are also configured to present to the professional images that may be received from database 40 or any other information.

The HUD includes a processor 724, mounted in a processor housing 726, which operates elements of the HUD. Processor 724 typically communicates with processor 26 via an antenna 728, although in some embodiments processor 724 may perform some of the functions performed by processor 26, and in other embodiments may completely replace processor 26.

Mounted on the front of HUD 700 is a flashlight 732. The flashlight projects visible spectrum light onto objects so that professional 22 is able to clearly see the objects through displays 720. Elements of the head-up display are typically powered by a battery (not shown in the figure) which supplies power to the elements via a battery cable input 736.

HUD 700 is held in place on the head of professional 22 by a head strap 740, and the professional may adjust the head strap by an adjustment knob 744.

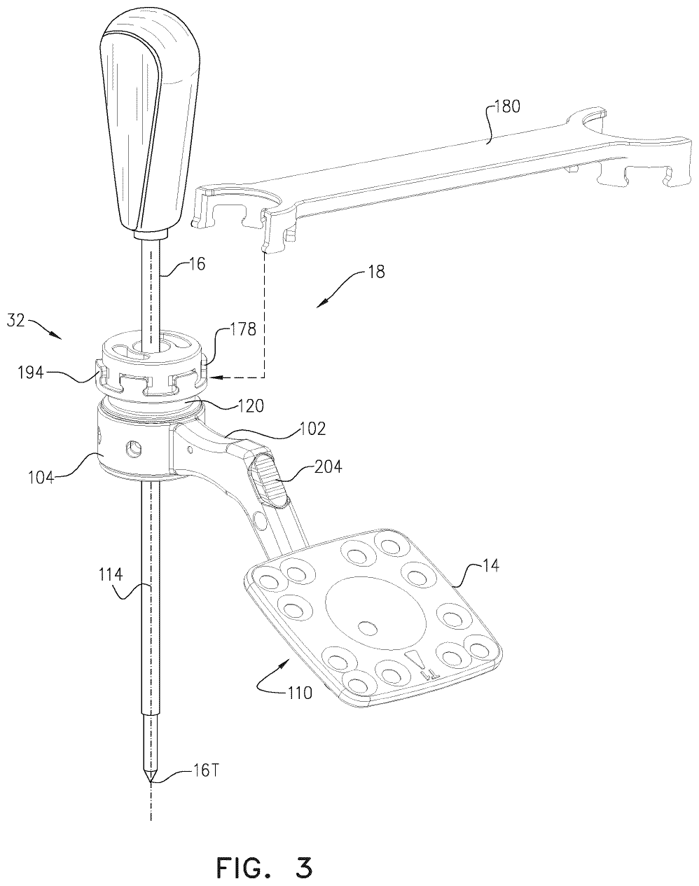

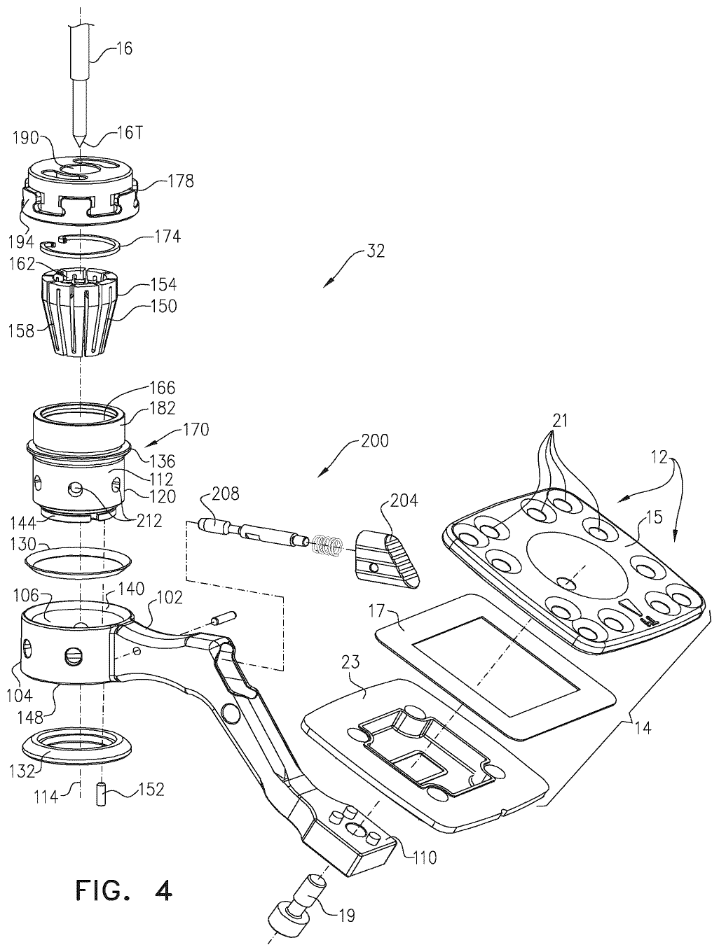

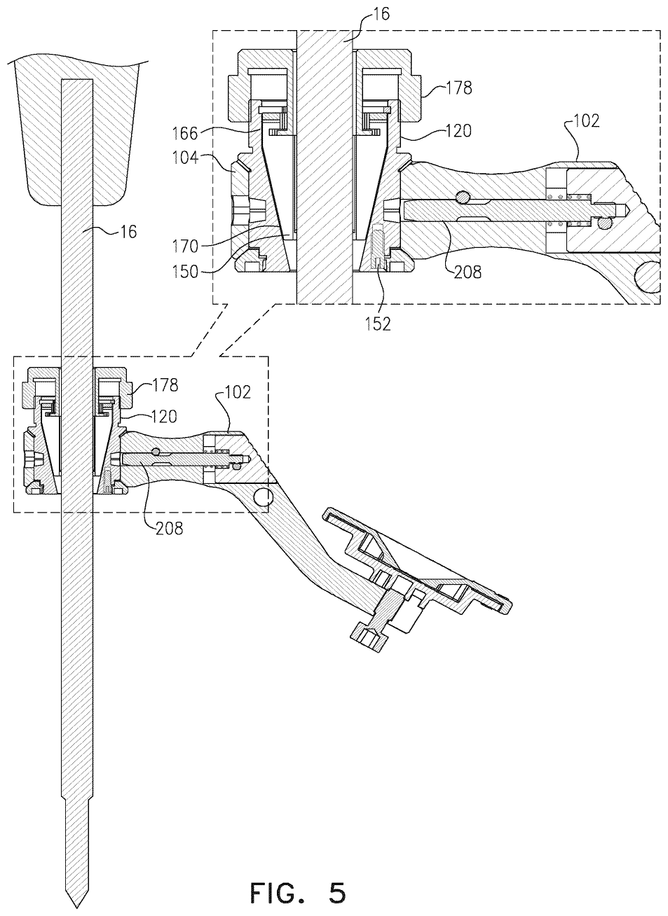

FIG. 3 is a schematic diagram illustrating exemplary tool adapter 18 and tool 16, FIG. 4 is a schematic exploded view of the tool adapter and the tool, and FIG. 5 is a schematic cross-section of the tool adapter and the tool, according to an embodiment of the present disclosure. Tool adapter 18 is formed from an adapter arm 102, which terminates at an upper or distal end of the arm in a circular coupling 104, herein also termed a circular opening 104, and which terminates at a lower or proximal end of the arm at a connection 110 which fixedly connects the arm to marker 14. A marker similar to marker 14 is described in U.S. patent application Ser. No. 16/199,281 to Messinger et al., which is incorporated herein by reference.

In a disclosed embodiment arm 102 is formed as a single unit. However, in other embodiments arm 102 may be formed as two or more units fixedly connected together. For example, opening 104 may be formed as a separate unit from the remainder of arm 102, and the opening may be fixedly connected by any convenient means to the remainder of the arm.

A tool grip 32 fixedly holds tool 16, as is described in more detail below, and the tool grip is rotatingly connected to circular opening 104, so that tool 16 rotates with respect to the opening as the grip rotates.

Circular opening 104 comprises an internal cylindrical surface 106 having a diameter that is typically in a range 10 mm-50 mm, and the surface defines an axis of symmetry 114 of the opening. Tool Grip 32 comprises a rigid collet holder 120, having an external cylindrical surface 112 with a diameter slightly smaller than that of internal cylindrical surface 106, that fits into opening 104. Except as described below, collet holder 120 is able to freely rotate within opening 104.

After assembling collet holder 120 into opening 104, the holder is held, so that it cannot translate along axis 114, by an upper circular washer 130 and a lower locking nut 132. Locking nut 132 acts as a washer due to its geometry and the material from which it is formed. The washer and nut are typically formed from a low friction material such as PEEK, and act as friction bearings, preventing the holder from translating along axis 114 while allowing the holder to rotate about the axis. Upper washer 130 is held in place between a circular protruding ridge 136 fixedly formed on surface 112 and an upper edge 140 of opening 104.

Lower locking nut 132 is threaded and is configured to mate with a threaded lower region 144 of holder 120, and is dimensioned to mate with a lower edge 148 of opening 104. The lower locking nut is held in place in region 144 by adhesive between the nut and the region, and with a set screw 152 that screws into region 144. Systems other than set screw 152 for holding the locking nut in place, such as a dowel pin, will be familiar to those having ordinary skill in the art, and all such systems are assumed to be comprised within the scope of the present disclosure.

As is described below, in some embodiments the locking nut may be used to limit, in a controlled manner, the freedom of rotation of arm 102 around collet holder 120.

A collet 150 fits within collet holder 120. Collet 150 has an upper external portion 154 that is cylindrical, and a lower external portion 158 that is conical. Collet 150 also has a central cylindrical aperture 162 that is dimensioned to accept tool 16 when the collet is uncompressed, and to grip the tool when the collet is compressed. An embodiment of the disclosure comprises a set of collets, each collet of the set being able to accept tools with a range of diameters.

Collet holder 120 has an internal upper cylindrical surface 166 and a lower internal conical surface 170, the two internal surfaces being dimensioned to mate with the external surfaces of collet 150 (FIG. 5).

Collet 150 is held in place within the collet holder by a retaining spring 174, the retaining spring being sprung against a dedicated groove in upper cylindrical surface 166. A collet fastening head 178 is configured to screw onto a threaded outer surface 182 of collet holder 120. Fastening head 178 is configured internally to mate with spring 174, so that when the head is screwed on surface 182 so as to move towards the collet holder, it pushes on spring 174 and thus pushes and compresses collet 150.

An internal cylindrical tube 190 is fixed to an upper surface of collet fastening head 178, the tube having an internal diameter larger than the external diameter of cylindrical tool 16. An outer surface of fastening head 178 is configured to be able to be gripped by wrench 180 so that the wrench is able to turn the fastening head. By way of example, in one embodiment the outer surface has protuberances 194, and wrench 180 mates with the protuberances so that the wrench is able to rotate the fastening head.

Once tool 16 is positioned within adapter 18, so that it traverses tube 190, rotation of collet fastening head 178 pushes on a flat edge of collet 150 causing the collet to be compressed, and thus to grip tool 16. It will be appreciated that while the tool is gripped by the collet, adapter arm 102 is able to rotate around the tool.

From review of the above description, it will be appreciated that tool grip 32 comprises collet holder 120, and other elements described herein that are connected to the collet holder and collet 150, apart from arm 102 and tool 16.

In some embodiments arm 102 comprises a locking mechanism 200. Mechanism 200 comprises an operating button 204, which is configured to toggle, via a spring and a connecting rod, a pin 208 of the mechanism to mate with or disengage from one of holes 212 in collet holder surface 112. Use of the locking mechanism facilitates attachment of tool 16 to tool adapter 18, by preventing the free rotation of collet holder 120 around the center axis of the tool while wrench 180 rotates fastening head 178.

In some embodiments circular opening 104 has holes that align with holes 212. The holes in opening 104 may be used by professional 22 to align mechanism 200 with holes 212, and may also facilitate flushing of liquids and avoidance of residual liquid when adapter 18 is cleaned.

As stated above marker 14 is fixedly attached to lower connection 110. In an illustrated embodiment marker 14 comprises optical elements 12 formed on marker 14. Elements 12 may be formed as a plurality of apertures 21 in an upper marker section 15, the apertures being backed by a retroreflective sheet 17 that is held in place with respect to the apertures by a lower marker section 23 fixed to the upper marker section. Marker 14 is fixedly connected by a screw 19 to lower connection 110.

Elements 12 are typically configured to have no rotational or reflective axis of symmetry, so that processor 26 is able to use the image of marker 14, including the images of elements 12 acquired by image capturing device 68 and/or camera 72 of assembly 24, to track the marker, i.e., to determine the location and orientation of the marker in a frame of reference defined by the assembly.

In some embodiments, typically on production of adapter 18, locking nut 132 may be used to limit, in a controlled manner, the freedom of rotation of arm 102 around collet holder 120. In this case the locking nut acts as a torque limiting device. (With no limitation on its rotation, during a procedure professional 22 may inadvertently cause the arm to rotate. While, as described herein, inadvertent rotation of the arm does not affect the functioning of system 20, such rotation may be undesired.)

With no limit on the arm rotation, it will be understood that once tool 16 has been clamped by collet 150 to collet holder 120, and the tool is held horizontal, the weight of arm 102, i.e. the force on the arm due to gravity, will cause the arm to rotate about axis 114 to a vertical “6 o'clock” position. As stated above, locking nut 132 may be adjusted to counteract the force of gravity, so that when tool 16 is held horizontal, arm 102 does not rotate but is also horizontal, in a “3 o'clock” position. The locking nut acts to provide a countervailing torque to the torque generated by gravity. The countervailing torque, herein also termed a threshold torque, does not completely prevent rotation of arm 102, but reduces inadvertent rotation of the arm, since rotation only occurs when the threshold torque is exceeded.

It will be understood that the threshold torque provided by locking nut 132 prevents adapter 18 from rotating due to the force of gravity, but does not apply too much friction that could prevent smooth rotation of tool 16 by professional 22. For an arm and marker having a mass of 80 g, and a center of mass 100 mm from axis 114, the threshold torque applied to arm 192 is approximately 8 N-cm.

In addition to acting as a torque limiting device, locking nut 132 also acts as a controller of the amount of total indicated runout (TIR) of a tool gripped by adapter 18. If locking nut 132 is set to have a threshold torque of 8 N-cm, then the TIR is approximately 60 microns or less.

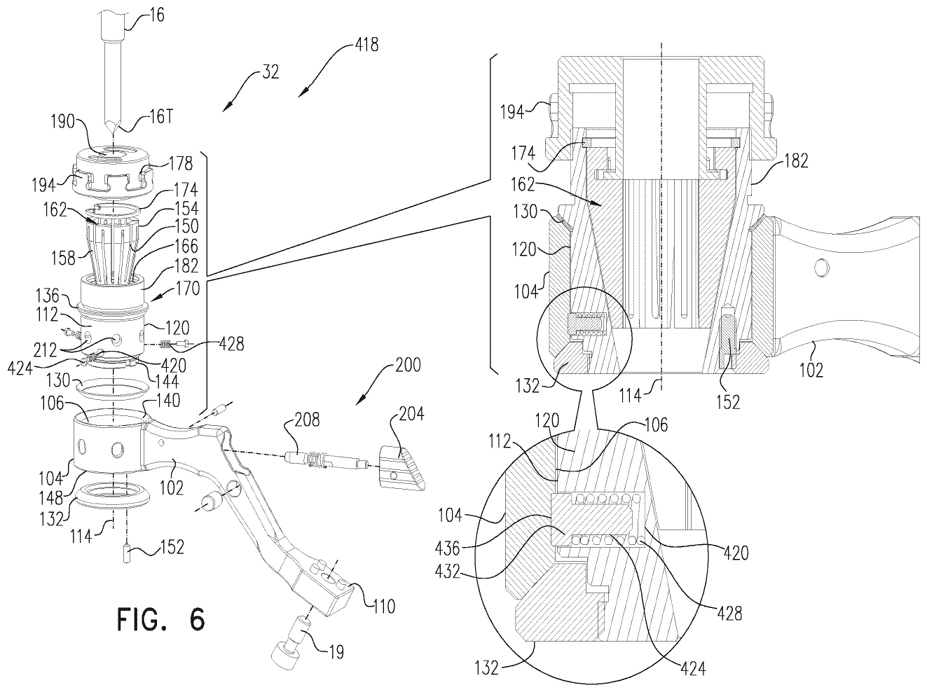

FIG. 6 is a schematic diagram illustrating an exemplary tool adapter 418 and tool 16, according to another embodiment of the present disclosure. Apart from the differences described below, the operation of adapter 418 is generally similar to that of adapter 18 (FIG. 1 5), and elements indicated by the same reference numerals in both adapters 18 and 418 are generally similar in construction and in operation.

In contrast to adapter 18, in adapter 418 three substantially similar blind holes 420 are formed in collet holder 120. Blind holes 420 are distributed symmetrically about axis 114, and are orthogonal to the axis.

Three substantially similar springs 428 are inserted into holes 420, and three substantially similar pins 424 are inserted into the springs. Each pin 424 has a terminal shoulder 432 which has an outer diameter that fits to the diameter of its hole 420.

In addition, the springs and pins are dimensioned so that after insertion each spring lower end contacts the base of its blind hole, the spring upper end contacts shoulder 432, and an exposed surface 436 of the shoulder slightly protrudes from surface 112, as illustrated in FIG. 6.

After assembly of adapter 418, each surface 436 pushes against internal surface 106 of opening 104 with a force that is a function of parameters of spring 428, i.e., the spring constant of the spring as well as its changed length. The pushing force in turn generates a frictional force on opening 104 when the opening rotates, or attempts to rotate, about axis 114, and the frictional force provides the threshold torque referred to above.

It will be understood that the threshold torque may be set to the value of 8 N-cm cited above, or to a value above or below this value, by selecting a spring with appropriate parameters, as well as by selecting the materials from which pin 424 and opening 104 are formed. Thus, any desired threshold torque may be implemented by these selections, without undue experimentation.

To assemble tool grip 32, springs 428 and pins 424 are first positioned in their blind holes. Opening 104 of the holder arm are then slid over collet holder 120, and over pin shoulders 432. Finally, locking nut 132 is screwed onto holder 120.

In contrast to adapter 18, wherein, as described above, locking nut 132 acts to set values of TIR and the threshold torque, in adapter 418 the two parameters can be set independently. I.e., in adapter 418 locking nut 132 is used to set the value of TIR, and springs 428 and pins 424 are used to set the value of the threshold torque.

The description above assumes that there are three sets of springs 428 and pins 424 distributed symmetrically about axis 114. However, it will be understood that any other convenient plurality of springs and pins distributed symmetrically about the axis, such as two, four, or five springs and pins, may be used as a torque limiting device.

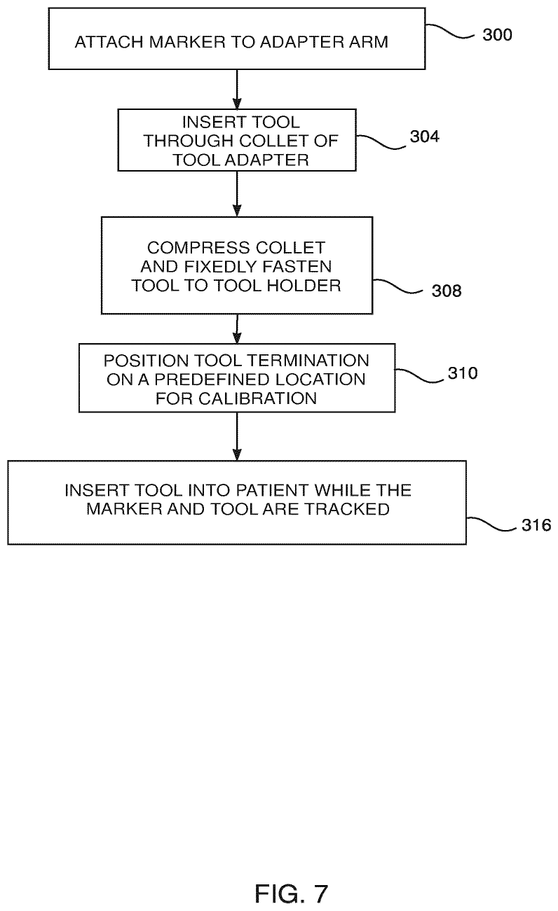

FIG. 7 is a flowchart of steps performed in using the disclosed tool adapters (e.g., tool adapter 18 or tool adapter 418), with augmented reality system 20 (FIG. 1), according to an embodiment of the present disclosure. The steps may be performed by a user of the adapters, such as professional 22.

In an initial step 300, a marker, such as marker 14, is attached to the tool adapter. For example, marker 14 may be attached to connection 110 of arm 102 of the tool adapter. As another example, tool adapter 418 is assembled, as described above with reference to FIGS. 3, 4, 5 and 6. The assembly typically includes adjusting locking nut 132 to provide a pre-determined TIR, and positioning pins 424 and springs 428 as described above. In some embodiments, initial step 300 is not required since the marker may be already attached to or incorporated in the tool adapter.

In a tool insertion step 304, a cylindrical tool, such as tool 16, is inserted through an aperture (e.g., aperture 162) of a collet of the adapter (e.g., collet 150). Since the tool is to be used in the medical procedure referred to above on a patient (e.g., patient 30), the tool is inserted so that the tip (e.g., tip 16T) of the tool 16 below the level of the marker.

In a collet activation step 308, the collet is compressed. The compression causes the collet to grip the tool. For example, wrench 180 is used to rotate collet fastening head 178 so as to compress collet 150, the compression causing the collet to grip tool 16. While wrench 180 is being used, locking mechanism 200 should be activated to lock collet holder 120 in place, as described above, so as to facilitate the compression of collet 150.

Steps 300-308 provide a method for assembling the tool and tool adapter and/or making the tool ready to be used with or in the frame of an image-guided system and during a medical procedure. Step 310 below refers to a calibration step performed via the image-guided system and step 316 refers to the use of the adaptor to track the tool during the medical procedure or surgery.

As described above, processor 26 is able to track or access or receive tracking information with respect to marker 14. Thus, in a calibration step 310, professional 22 positions tool termination 16T on a predefined location, and processor 26 acquires or accesses (e.g., via camera 68 and/or 73) an image of marker 14. From the acquired image, the processor calculates a position of the marker, i.e., its location and orientation, and forms a vector correspondence between the marker position and tool termination 16T and the orientation of tool 16. I.e., the processor transforms the marker position to tool termination 16T and to the direction of axis 114. It will be understood that the vector correspondence is unchanged if arm 102 rotates around axis 114, since the marker is fixed to the arm.

In a tool insertion step 316, tool, e.g. tool 16, is inserted into a patient (e.g., patient 30) while the marker (e.g., marker 14) is tracked (e.g., via camera 68 and/or 73 and processor 26). The tracking of the marker provides the processor with the location and orientation, i.e., the marker position, of the marker. From the marker position the processor is able to find, using the vector correspondence found in or following step 310, the location of the tool termination and the orientation of the tool. The processor is able to use the orientation of the tool and the location of the tool termination (or tip) in presenting correctly registered images in a near-eye assembly or head-mounted assembly like assembly 24.

In one embodiment tool 16 comprises a screwdriver, which is inserted into patient 30 so that professional 22 is able to adjust a pedicle screw. It will be appreciated that while the screwdriver is turned, processor 26 is still able to track the screwdriver orientation and its termination, using the vector correspondence found in calibration step 310, so long as marker 14 is tracked.

Alternatively or additionally, during the procedure positioning marker 14 may obstruct the professional's view of the patient, and/or the view of the patient as acquired by devices 68 and/or camera 72. In any of these events, and according to some embodiments, professional 22 may rotate arm 102 about axis 114, applying a torque greater than the threshold torque applied in step 300, so that marker 14 no longer obstructs the view, but while the marker is continuously tracked. Since marker 14 continues to be tracked, the processor is able to use the new tracked location of the marker to continue tracking tool 16 and termination 16T of the tool, since the spatial relationships, i.e., the vector correspondence, between the tool and the marker, and between the tool termination and the marker, are unchanged by the tool adapter rotation about axis 114.

Those having ordinary skill in the at will appreciate that the order of steps 300-316 described above is but one example of possible orders of the steps, and that the steps may be performed in a different order from that given here. For example, initial step 300 may be performed after collet activation step 308. All such orders are assumed to be comprised within the scope of the present disclosure.

While the description above has used as an example a tool that may be rotated about its axis, e.g., a screwdriver, in the tool adapters described herein, it will be understood that tools used in the adapters do not need to rotate. Thus, a tool used in the adapters may be fixed or non-rotatable about a tool axis.

It will thus be appreciated that the embodiments described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

What is claimed is:

1. A system, comprising:
a tool adapter comprising:
an adapter arm having a proximal end and a distal end comprising an opening;
a tool grip configured to retain a tool, the tool grip being rotatingly connected to the opening so as to permit rotation of the tool grip with respect to the adapter arm when assembled; and
a position marker connected to the proximal end;
a processor configured for tracking the tool adapter; and
a head-up display coupled to the processor, the head-up display comprising:
one or more augmented reality displays configured for displaying an augmented reality image including the position marker.

2. The system of claim 1, wherein the tool grip comprises a collet housed in a collet holder, and wherein when the tool is inserted through the tool grip, the collet is configured, when compressed, to grip the tool.

3. The system of claim 2, wherein the collet holder is held by the opening, and is configured to be unable to move along an axis orthogonal to the opening.

4. The system of claim 2, wherein the tool adapter further comprises a collet fastening head configured to screw onto the collet holder so as to implement the compression of the collet.

5. The system of claim 1, wherein the position marker is fixedly connected to a connection of the proximal end in a preset spatial relationship with an axis orthogonal to the opening, the position marker comprising optical elements enabling the position marker to be tracked spatially, so that tracking of the position marker provides tracking of the tool retained by the tool grip.

6. The system of claim 1, wherein the tool adapter further comprises a torque limiting device coupled to the tool grip and configured to apply a preset threshold torque to the tool grip so that rotation of the tool grip is permitted when the preset threshold torque is exceeded.

7. The system of claim 6, wherein the torque limiting device comprises a plurality of pins retained within the tool grip and configured to exert force against the opening.

8. The system of claim 1, wherein the tool adapter further comprises a total indicated runout (TIR) controller coupled to the tool grip and configured to apply a threshold torque to control an amount of TIR of the tool.

9. The system of claim 1, wherein the head-up display further comprises:
an optics housing comprising:
an infrared camera; and
an infrared transparent window; and
one or more infrared projectors within the optics housing.

10. The system of claim 9, wherein the one or more augmented reality displays are on the optics housing, and configured to display a visual element stored via a database in data communication with the processor.

11. The system of claim 1, wherein the head-up display further comprises an antenna configured for data communication with the processor.

12. The system of claim 1, wherein the head-up display further comprises a flashlight configured to project visible spectrum light onto an object viewable through the one or more augmented reality displays.

13. The system of claim 1, wherein the head-up display further comprises an energy storage system configured for powering one or more elements of the head-up display.

14. The system of claim 1, wherein the head-up display further comprises a head strap configured for holding the head-up display in place on a user's head, the head strap comprising an adjustment knob configured for adjusting the head strap.

15. The system of claim 1, wherein:
the proximal end terminates with a connection,
the opening comprises a circular opening having a center and defining an axis orthogonal to the circular opening and passing through the center,
the tool grip is configured to retain the tool along the axis, and
the tool grip is rotatingly connected to the circular opening so as to permit rotation of the tool grip about the axis when assembled.

16. A head-up display, comprising:
a processor configured for tracking a tool adapter, wherein the tool adapter comprises:
an adapter arm having a proximal end terminating with a connection and a distal end comprising an opening, the opening defining an axis orthogonal to the opening,
a tool grip configured to retain a tool along the axis, the tool grip being rotatingly connected to the opening so as to permit rotation of the tool grip with respect to the adapter arm and about the axis when assembled, and
a position marker connected to the connection of the proximal end; and
one or more augmented reality displays configured for displaying an augmented reality image including the position marker.

17. The head-up display of claim 16, wherein:
the opening comprises a circular opening having a center, and
the axis passes through the center.

18. The head-up display of claim 16, further comprising:
an optics housing comprising:
an infrared camera; and
an infrared transparent window; and
one or more infrared projectors within the optics housing.

19. The head-up display of claim 18, wherein the one or more augmented reality displays are on the optics housing, and configured to display a visual element stored via a database in data communication with the processor.

20. The head-up display of claim 16, further comprising a flashlight configured to project visible spectrum light onto an object viewable through the one or more augmented reality displays.
